(19) United States
(12) Patent Application Publication
(10) Pub. No.: US 2024/0399027 A1
(43) Pub. Date: Dec. 5, 2024

(54) BIO-INK, APPLICATION THEREOF, PENILE CORPUS CAVERNOSUM PROSTHESIS AND PREPARATION METHOD THEREOF

(71) Applicant: South China University of Technology, Guangzhou (CN)

(72) Inventors: Xuetao SHI, Guangzhou (CN); Tan YE, Guangzhou (CN); Zhenxing WANG, Guangzhou (CN)

(73) Assignee: South China University of Technology, Guangzhou (CN)

(21) Appl. No.: 18/667,863

(22) Filed: May 17, 2024

(30) Foreign Application Priority Data
Jun. 2, 2023 (CN) 2023106529677

(51) Int. Cl. A61L 27/48; A61L 27/52; B33Y 10/00; B33Y 70/00

(52) U.S. Cl. CPC A61L 27/48; A61L 27/52; B33Y 10/00; B33Y 70/00

(57) ABSTRACT

The disclosure belongs to the technical field of biomedical materials, and provides a bio-ink, application thereof, a penile corpus cavernosum prosthesis and a preparation method thereof. The bio-ink provided by the disclosure includes a polymer-based material, an initiator, a crosslinking agent, a light absorber, and water. The bio-ink provided by the disclosure can undergo a gelation reaction under ultraviolet light irradiation, and an obtained hydrogel matrix material can have an elongation at break reaching 200-600%, so as to be applied to 3D printing. The disclosure further provides a penile corpus cavernosum prosthesis. For the penile corpus cavernosum prosthesis provided by the disclosure, with biocompatible hydrogel as a matrix material, an anticoagulant layer obtained by coating can effectively reduce the risk of thrombosis and has good safety and stability. The penile corpus cavernosum prosthesis of the disclosure has bionic vascular sinus cavity and bionic white membrane structures.

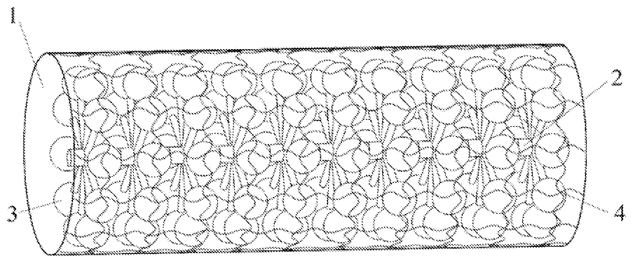

### CROSS-REFERENCE TO RELATED APPLICATIONS

The application claims priority to Chinese patent application No. 2023106529677, filed on Jun. 2, 2023, the entire contents of which are incorporated herein by reference.

### TECHNICAL FIELD

The disclosure relates to the technical field of biomedical materials, and in particular to a bio-ink, application thereof, a penile corpus cavernosum prosthesis and a preparation method thereof.

### BACKGROUND

A penis is an important reproductive and urinary organ of a man. Normal erection of the penis is an important prerequisite for completion of sexual life. Erectile dysfunction (ED), caused by many factors, of the penis can lead to inability to produce and maintain normal erection of the penis. Except affecting the normal sexual life, the ED can further endanger mental health of a patient.

For a patient with a more severe organic ED, implantation of a penile prosthesis is a common and effective method. A traditional penile prosthesis is a three-piece penile prosthesis, which generally includes an expandable cylindrical part, a sac and a pump. By pumping water in the sac into the cylinder, the cylinder expands to simulate congestion of a corpus cavernosum, making the penis appear to be close to a physiological erection state. As it is closer to flaccid and erection states of the human penis, an operation is simple, convenient and efficient, and the three-piece penile prosthesis has become a mainstream trend of prosthesis development. Among them, the expandable cylinder as a corpus cavernosum prosthesis is a core part of such prosthesis, having high requirements on a material and a structure. At present, commonly used materials are materials with good biocompatibility such as silicone rubber. However, due to a difference in stiffness, poor affinity and other heterogeneity challenges, it will lead to discomfort in an early stage of implantation, and damage the remaining corpus cavernosum and a function thereof. In addition, a size of the prosthesis will also affect comfort and functionality. Current products have problems such as poor customization and a high price.

### SUMMARY

A purpose of the disclosure is to overcome the problems in the prior art, and provides a bio-ink, application thereof, a penile corpus cavernosum prosthesis and a preparation method thereof.

In order to achieve the above purpose of the disclosure, the disclosure provides the following technical solutions.

The disclosure provides a bio-ink, prepared from the following raw materials in percentage by mass:
5-60% of a polymer-based material, 0.01-1% of an initiator, 0.05-10% of a crosslinking agent, 0.01-1% of a light absorber, and the balance of water.

Preferably, the polymer-based material is one or more of gelatin, chitosan, sodium alginate, agarose, hyaluronic acid, silk fibroin, dextran, polyvinyl alcohol, polyethylene glycol, acrylamide, acrylic acid, N-isopropylacrylamide, methacrylic acid, benzyl methacrylate, polyvinyl alcohol methacryloyl, 2-hydroxyethyl methacrylate, and N-vinylpyrrolidone.

The initiator is one or more of 2-hydroxy-4-(2-hydroxyethoxy)-2-methylpropiophenone, lithium phenyl-2,4,6-trimethylbenzoylphosphinate, benzophenone, and 2,4-dimethylthioxanthone.

Preferably, the crosslinking agent is one or more of N, N-methylenebisacrylamide, poly (ethylene glycol) diacrylate, and 4-arm poly (ethylene glycol) diacrylate.

The light absorber is one or more of hydroquinone, 1-(4-sulfophenyl)-4-(4-sulfophenylazo)-5-pyrazolone-3-carboxylic acid trisodium salt, 2,2′-dihydroxy-4,4′-dimethoxybenzophenone-5, and 5′-sodium disulfonate.

The disclosure further provides application of the bio-ink in 3D printing.

The disclosure further provides a penile corpus cavernosum prosthesis, including a main body hydrogel structure, an internal bionic vascular sinus cavity structure, and an external bionic white membrane structure.

The main body hydrogel structure is obtained by 3D printing with the bio-ink.

Preferably, the internal bionic vascular sinus cavity includes a cylindrical cavity structure and a spherical cavity structure.

A channel of the cylindrical cavity structure has a length of 1-100 mm, and a diameter of 0.1-10 mm.

The spherical cavity structure has a diameter of 0.5-5 mm.

Preferably, a fiber material of the external bionic white membrane structure is high density polyethylene fibers.

Each fiber of the external bionic white membrane structure has a diameter of 0.05-1 mm, and there are 10-40 fibers.

The disclosure further provides a preparation method for the penile corpus cavernosum prosthesis, including the following steps:
(1) performing 3D printing and curing on bio-ink to obtain a main body model structure;
(2) embedding fibers into an outer surface of the main body model structure, and then performing light curing to obtain a main body model with a bionic white membrane structure; and
(3) performing crosslinking and coating on the main body model with the bionic white membrane structure to obtain the penile corpus cavernosum prosthesis.

Preferably, in step (1), for 3D printing, a printing resolution is 10-50 μm, a printing speed is 1-60 s/layer, a printing power is 600-1000 w, and a height of each layer is 40-60 μm.

In step (1), for curing, a wavelength of ultraviolet light is 280-450 nm, and a time lasts for 0.5-2 h.

In step (1), a trench of the main body model structure has a width of 0.1-2 mm, and a depth of 0.2-4 mm.

In step (2), for light curing, a wavelength of ultraviolet light is 280-450 nm, and a time lasts for 5-10 min.

Preferably, in step (3), a crosslinking solution for crosslinking is one or more of a glutaraldehyde solution, a 1-ethyl-(3-dimethylaminopropyl) carbodiimide/N-hydroxysuccinimide solution, a genipin solution, a calcium chloride solution, and a sodium hydroxide solution.

A mass percentage of the crosslinking solution is 0.1-20%.

In step (3), a crosslinking time lasts for 5-30 h.

In step (3), coating is to load poly-L-lysine and heparin sodium in sequence.

A coating thickness is 1-10 μm.

The disclosure provides a bio-ink, including a polymer-based material, an initiator, a crosslinking agent, a light absorber, and water. The bio-ink provided by the disclosure can undergo a gelation reaction under ultraviolet light irradiation, and an obtained hydrogel matrix material can have an elongation at break reaching 200-600%, so as to be applied to 3D printing.

The disclosure further provides a penile corpus cavernosum prosthesis. For the penile corpus cavernosum prosthesis provided by the disclosure, with biocompatible hydrogel as a matrix material, the mechanical properties are compatible with tissues, and the safety and the stability are good. An anticoagulant layer obtained by coating can effectively reduce the risk of thrombosis.

The penile corpus cavernosum prosthesis of the disclosure has bionic vascular sinus cavity and bionic white membrane structures. Expansion with water injection corresponds to an erection state, which can make a penis reach a hardness and a size required for sexual life; and recovery with pressure relief corresponds to a flaccid state with reduced size and modulus, so that the penis maintains a more normal appearance.

An initial size of the penile corpus cavernosum prosthesis of the disclosure can be customized by modeling, and a size of the erection and expansion state can be regulated by the bionic white membrane structure as needed. The prepared prosthesis can meet the requirements of personalized customization of a patient. 3D printing has a high precision structure, and is fast in formation and low in cost.

### BRIEF DESCRIPTION OF DRAWINGS

The FIGURE is a schematic diagram of a penile corpus cavernosum prosthesis prepared in embodiment 1.

### DETAILED DESCRIPTION OF THE EMBODIMENTS

The disclosure provides a bio-ink, prepared from the following raw materials in percentage by mass:

5-60% of a polymer-based material, 0.01-1% of an initiator, 0.05-10% of a crosslinking agent, 0.01-1% of a light absorber, and the balance of water.

In the disclosure, a mass percentage of the polymer-based material is 5-60%, preferably, 10-55%, further preferably, 15-50%, and more preferably, 20-40%.

In the disclosure, a mass percentage of the initiator is 0.01-1%, preferably, 0.1-0.9%, further preferably, 0.2-0.8%, and more preferably, 0.4-0.6%.

In the disclosure, the polymer-based material is preferably one or more of gelatin, chitosan, sodium alginate, agarose, hyaluronic acid, silk fibroin, dextran, polyvinyl alcohol, polyethylene glycol, acrylamide, acrylic acid, N-isopropylacrylamide, methacrylic acid, benzyl methacrylate, polyvinyl alcohol methacryloyl, 2-hydroxyethyl methacrylate, and N-vinylpyrrolidone.

In the disclosure, the polyvinyl alcohol methacryloyl is prepared by the following method:
(1) mixing the polyvinyl alcohol, 4-dimethylaminopyridine, glycidyl methacrylate, and dimethyl sulfoxide to obtain a reaction solution; and
(2) mixing the reaction solution with acetone to obtain the polyvinyl alcohol methacryloyl.

In the disclosure, in step (1), a mass-volume ratio of the polyvinyl alcohol, the 4-dimethylaminopyridine, the glycidyl methacrylate, and the dimethyl sulfoxide is preferably (5-15 g):(0.2-0.25 g):(0.2-0.4 mL):(90-110 mL), further preferably, (6-14 g):(0.21-0.24 g):(0.25-0.35 mL):(95-105 mL); and more preferably, (8-12 g):(0.22-0.23 g):(0.28-0.32 mL):(98-102 mL).

In the disclosure, in step (1), a mixing temperature is preferably 50-70° C., further preferably, 55-65° C., and more preferably, 58-62° C.; and a mixing time preferably lasts for 20-28 h, further preferably, 21-27 h, and more preferably, 22-26 h.

In the disclosure, a volume ratio of the acetone to the dimethyl sulfoxide in step (2) is preferably (400-600):(90-110), further preferably, (450-550):(95-105), and more preferably, (480-520):(98-102).

In the disclosure, in step (2), after uniform mixing, precipitates are obtained by suction filtration, washed with the acetone, and subjected to vacuum oven drying to obtain the polyvinyl alcohol methacryloyl.

In the disclosure, the polymer-based material can improve the biocompatibility of an overall structure and regulate the mechanical properties of the structure.

In the disclosure, the initiator is one or more of 2-hydroxy-4-(2-hydroxyethoxy)-2-methylpropiophenone, lithium phenyl-2,4,6-trimethylbenzoylphosphinate, benzophenone, and 2,4-dimethylthioxanthone.

In the disclosure, the initiator absorbs energy of light of a specific wavelength, to produce free radicals and initiate photopolymerization.

In the disclosure, a mass percentage of the crosslinking agent is 0.05-10%, preferably, 1-9%, further preferably, 2-8%, and more preferably, 4-6%.

In the disclosure, a mass percentage of the light absorber is 0.01-1%, preferably, 0.1-0.9%, further preferably, 0.2-0.8%, and more preferably, 0.4-0.6%.

In the disclosure, the crosslinking agent is preferably one or more of N, N-methylenebisacrylamide, poly (ethylene glycol) diacrylate, and 4-arm poly (ethylene glycol) diacrylate.

In the disclosure, the light absorber is preferably one or more of hydroquinone, 1-(4-sulfophenyl)-4-(4-sulfophenylazo)-5-pyrazolone-3-carboxylic acid trisodium salt, 2,2′-dihydroxy-4, 4′-dimethoxybenzophenone-5, and 5′-sodium disulfonate.

In the disclosure, the light absorber can competitively absorb ultraviolet light and reduce a decrease in a printing resolution caused by light transmission or scattering.

The disclosure further provides application of the bio-ink in 3D printing.

The disclosure further provides a penile corpus cavernosum prosthesis, including a main body hydrogel structure, an internal bionic vascular sinus cavity structure, and an external bionic white membrane structure.

The main body hydrogel structure is obtained by 3D printing with the bio-ink.

In the disclosure, the internal bionic vascular sinus cavity structure includes a cylindrical cavity structure and a spherical cavity structure.

In the disclosure, a channel of the cylindrical cavity structure has a length preferably of 1-100 mm, further preferably, 10-90 mm, and more preferably, 20-80 mm, and has a diameter preferably of 0.1-10 mm, further preferably, 1-9 mm, and more preferably, 2-8 mm.

In the disclosure, the cylindrical cavity structure is further connected to a branch tubular structure; and a pore canal of the branch tubular structure has a diameter preferably of 0.5-1 mm, further preferably, 0.6-0.9 mm, and more preferably, 0.7-0.8 mm, and has a length preferably of 4-8 mm, further preferably, 5-7 mm, and more preferably, 5.5-6.5 mm.

In the disclosure, the spherical cavity structure has a diameter preferably of 0.5-5 mm, further preferably, 1-4 mm, and more preferably, 2-3 mm.

In the disclosure, the cylindrical cavity structure and the spherical cavity structure are connected to each other, to construct the bionic vascular sinus cavity structure. During real erection of the penile, a large amount of blood flows into a cavernous artery, making a cavernous sinus expand to compress a reflux vein, and then achieve congestion and expansion.

In the disclosure, the bionic vascular sinus cavity structure communicates with the external at one end of the cylinder, to be used for connecting a water injection pipeline.

In the disclosure, a fiber material of the external bionic white membrane structure is preferably high density polyethylene fibers.

In the disclosure, each fiber of the external bionic white membrane structure has a diameter preferably of 0.05-1 mm, further preferably, 0.1-0.9 mm, and more preferably, 0.4-0.6 mm; and there are preferably 10-40 fibers, further preferably, 15-35 fibers, and more preferably, 20-30 fibers.

In the disclosure, the periphery of a corpus cavernosum is wrapped with a white membrane which has collagen fiber and elastic fiber structures. During penile erection, high modulus collagen fibers are stretched by curling, which limits a maximum strain of the white membrane to maintain hardness and a morphology of the penis during erection.

In the disclosure, the high density polyethylene fibers of the external bionic white membrane are in an orthogonal wavy shape; a trajectory curve of the high density polyethylene fibers is a controllable trigonometric function curve, which can play a role of strain limitation; and a trigonometric function of the orthogonal wavy trajectory curve is: y=1.375×sin (πx/27) or y=1.25×sin (πx/24).

In the disclosure, when the penile corpus cavernosum prosthesis expands with water injection, a length can reach 1.2-2.4 times the initial size, and a diameter can reach 1.2-1.8 times the initial size, which are close to a physiological corpus cavernosum erection deformation.

The disclosure further provides a preparation method for the penile corpus cavernosum prosthesis, including the following steps:
(1) performing 3D printing and curing on bio-ink to obtain a main body model structure;
(2) embedding fibers into an outer surface of the main body model structure, and then performing light curing to obtain a main body model with a bionic white membrane structure; and
(3) performing crosslinking and coating on the main body model with the bionic white membrane structure to obtain the penile corpus cavernosum prosthesis.

In the disclosure, a model is designed first. The model is in a cylindrical shape, has the bionic vascular sinus cavity structure inside and an orthogonal wavy trench structure on an outer surface. The trench structure is to accommodate subsequent high density polyethylene fibers, and the model is generated as needed. A light-curing 3D printer is used to import a model design file and perform 3D printing.

In the disclosure, in step (1), for 3D printing, a printing resolution is preferably 10-50 μm, further preferably, 15-45 μm, and more preferably, 20-30 μm; a printing speed is 1-60 s/layer, further preferably, 10-50 s/layer, and more preferably, 20-30 s/layer; a printing power is preferably 600-1000 w, further preferably, 700-900 w, and more preferably, 750-850 w; and a height of each layer is preferably 40-60 μm, further preferably, 45-55 μm, and more preferably, 48-52 μm.

In the disclosure, in step (1), for light curing, a wavelength of ultraviolet light is preferably 280-450 nm, further preferably, 300-400 m, and more preferably, 340-360 nm; and a time preferably lasts for 0.5-2 h, further preferably, 0.6-1.8 h, and more preferably, 1-1.4 h.

In the disclosure, a printed model needs to be cured, to increase a degree of photopolymerization, enhance structural strength, and reduce residues of unreacted materials.

In the disclosure, in step (1), a trench of a main body model structure has a width preferably of 0.1-2 mm, further preferably, 0.5-1.5 mm, and more preferably, 0.8-1.2 mm; and the trench has a depth preferably of 0.2-4 mm, further preferably, 0.5-3.5 mm, and more preferably, 1-2 mm.

In the disclosure, trench trajectory curves are all specific trigonometric function curves, corresponding to the polyethylene fiber trajectory in the bionic white membrane. A curve formula can be set according to a prosthesis size required after expansion.

In the disclosure, the high density polyethylene fibers are cut according to the size of the trench, buried into the trench, and knotted for fixing, and the excess fibers are cut off; the bio-ink without the light absorber is added to the trench for UV curing; and the polyethylene fibers are fixed to obtain a main body model with the bionic white membrane structure on the surface.

In the disclosure, in step (2), for light curing, a wavelength of ultraviolet light is preferably 280-450 nm, further preferably, 300-400 nm, and more preferably, 340-360 nm; and a time preferably lasts for 5-10 min, further preferably, 6-9 min, and more preferably, 7-8 min.

In the disclosure, in step (3), a crosslinking solution for crosslinking is preferably one or more of a glutaraldehyde solution, a 1-ethyl-(3-dimethylaminopropyl) carbodiimide/N-hydroxysuccinimide solution, a genipin solution, a calcium chloride solution, and a sodium hydroxide solution.

In the disclosure, when the 1-ethyl-(3-dimethylaminopropyl) carbodiimide/N-hydroxysuccinimide solution is selected as the crosslinking solution, a mass ratio of 1-ethyl-(3-dimethylaminopropyl) carbodiimide to N-hydroxysuccinimide is preferably (0.02-0.04):(0.05-0.15), further preferably (0.025-0.035):(0.06-0.14), and more preferably, (0.028-0.032):(0.08-0.12).

In the disclosure, a mass percentage of the crosslinking solution is preferably 0.1-20%, further preferably, 5-15%, and more preferably, 8-12%.

In the disclosure, the main body model with the bionic white membrane structure on the surface obtained in step (2) is soaked into the crosslinking solution to form chemical crosslinking and physical crosslinking such as ion crosslinking and hydrogen bonding, which further improves structural mechanical properties and structural stability.

In the disclosure, in step (3), a crosslinking time preferably lasts for 5-30 h, further preferably, 10-25 h, and more preferably, 15-20 h.

In the disclosure, in step (3), coating is to load poly-L-lysine and heparin sodium in sequence.

In the disclosure, in step (3), coating is performed after completion of crosslinking, and the anticoagulant layer is obtained by electrostatically adsorbing layers of self-assembled positively charged poly-L-lysine and negatively charged heparin sodium. The heparin sodium can effectively reduce platelet adhesion and local coagulation after implantation of the prosthesis.

In the disclosure, the crosslinked model is soaked into a poly-L-lysine solution and then into a heparin sodium solution, to complete a single cycle, and the anticoagulant layer is obtained after multiple cycles.

In the disclosure, a concentration of the poly-L-lysine solution is 0.5-1.5 mg/mL, further preferably, 0.6-1.4 mg/mL, and more preferably, 0.8-1.2 mg/ml; a soaking time in the poly-L-lysine solution preferably lasts for 0.5-1.5 h, further preferably, 0.6-1.4 h, and more preferably, 0.8-1.2 h; a concentration of the heparin sodium solution is preferably 5-15 mg/mL, further preferably, 6-14 mg/mL, and more preferably, 8-12 mg/mL; and a soaking time in the heparin sodium solution preferably lasts for 0.5-1.5 h, further preferably, 0.6-1.4 h, and more preferably, 0.8-1.2 h.

In the disclosure, coating is completed after multiple cycles to obtain the anticoagulant layer; and a coating thickness is preferably 1-10 μm, further preferably, 2-8 μm, and more preferably, 4-6 μm.

The technical solutions provided by the disclosure are described in detail below with embodiments, but they cannot be understood as limiting the protection scope of the disclosure.

### Embodiment 1

The bio-ink is prepared, wherein 20% of acrylamide, 10% of gelatin, 0.05% of N, N-methylenebisacrylamide, 0.5% of lithium phenyl-2,4,6-trimethylbenzoylphosphinate, 0.12% of 1-(4-sulfophenyl)-4-(4-sulfophenylazo)-5-pyrazolone-3-carboxylic acid trisodium salt, and the balance of water are stirred uniformly to prepare the bio-ink, and the bio-ink is stored in the dark.

A modeling software is used for modeling. A corpus cavernosum prosthesis has an external diameter of 15 mm and a length of 50 mm, and the cylindrical cavity structure has a length of 46 mm and a diameter of 3 mm; the branch tubular structure has a length of 6 mm and a diameter of 1 mm; a spherical hole has a diameter of 2.5 mm; the trajectory curve of the orthogonal wavy trench on the outer surface is: y=1.375×sin (xx/27); the trench has a width of 1 mm and a depth of 1 mm; and the software is used to slice up the model for storage.

A high-precision DLP printer is used for printing with a printing resolution of a light machine of 15 μm, a printing speed of 8 s/layer, a printing power of 800 w, and a height of each layer of 50 μm to obtain a model structure; and the printed model is placed in an ultraviolet box for curing at 365 nm for 1 h to obtain the main body model structure.

30 high density polyethylene fibers with the diameter of 0.8 mm are taken, buried into the trench on the surface, and knotted end to end for fixing, and excess fibers are cut off. The bio-ink without the 1-(4-sulfophenyl)-4-(4-sulfophenylazo)-5-pyrazolone-3-carboxylic acid trisodium salt is added to the trench for curing at 365 nm for 5 min to obtain the main body model with the bionic white membrane structure.

0.032 g of 1-ethyl-(3-dimethylaminopropyl) carbodiimide/N-hydroxysuccinimide and 0.134 g of N-hydroxysuccinimide are added to 50 mL of a 2-(N-morpholino) ethanesulfonic acid solution, wherein a pH value of the 2-(N-morpholino) ethanesulfonic acid solution is 6, and a molar concentration is 0.05 mol/L; the main body model with the bionic white membrane structure is soaked into the crosslinking solution for 24 h, so as to form chemical crosslinking between gelatin molecular chains; after completion of crosslinking, the main body model is soaked into 1 mg/mL poly-L-lysine solution for 1 h, and then soaked into 10 mg/mL heparin sodium solution for 1 h to complete a single cycle; and multiple cycles are performed until an anticoagulant layer of 2 μm thick on the surface is obtained, to obtain the penile corpus cavernosum prosthesis.

The schematic diagram of the penile corpus cavernosum prosthesis prepared in this embodiment is shown in the FIGURE, wherein 1 represents the main body hydrogel structure; 2 represents the cylindrical cavity structure of the internal bionic vascular sinus cavity structure; 3 represents the spherical cavity structure of the internal bionic vascular sinus cavity structure; and 4 represents the external bionic white membrane structure.

### Embodiment 2

This embodiment is different from embodiment 1 in that during preparation of the bio-ink, the polymer-based material used is 25% N-isopropylacrylamide and 2% sodium alginate; the printing speed is set as 15 s/layer; the main body model with the bionic white membrane structure is soaked into a calcium chloride solution with a mass percentage of 10% for 6 h.

### Embodiment 3

This embodiment is different from embodiment 1 in that during preparation of the bio-ink, the polymer-based material used is 10% polyvinyl alcohol methacryloyl and 5% gelatin; the printing speed is set as 12 s/layer; the main body model with the bionic white membrane structure is soaked into a glutaraldehyde solution with a mass percentage of 2% for 12 h.

In this embodiment, the polyvinyl alcohol methacryloyl is prepared as follows: 10 g of polyvinyl alcohol is dissolved into 100 ml of dimethyl sulfoxide; 0.22 g of 4-dimethylaminopyridine is added for dissolution; then 0.3 ml of glycidyl methacrylate is added for stirring in a water bath at 60° C. for 24 h; a reaction solution is added to 500 ml of acetone; and precipitates are obtained by suction filtration, clearly washed with the acetone, and subjected to vacuum oven drying to obtain the polyvinyl alcohol methacryloyl.

### Embodiment 4

This embodiment is different from embodiment 1 in that during preparation of the bio-ink, the polymer-based material used is 30% acrylic acid and 3% chitosan; and the printing speed is set as 20 s/layer.

### Embodiment 5

This embodiment is different from embodiment 1 in that when the modeling software is used for modeling, the trajectory curve of the orthogonal wavy trench on the outer surface is: y=1.25×sin (πx/24).

### Comparative Example 1

This comparative example is different from embodiment 1 in that the high density polyethylene fibers are not embedded.

### Comparative Example 2

This comparative example is different from embodiment 1 in that the main body model with the bionic white membrane structure is not soaked for crosslinking.

The penile corpus cavernosum prostheses prepared in embodiments 1-5 and comparative examples 1-2 are tested and evaluated. Results are shown as follows:

Corpus cavernosum prosthesis structures are prepared by using different material combinations in embodiments 1-4. Each prosthesis can uniformly expand after a certain amount of water is injected therein. By measuring size changes of the prosthesis before and after water injection, it can be seen that the diameter becomes 1.3 times an original diameter, and the length becomes 1.45 times an original length, and the erection state can be effectively simulated. It can be seen from embodiments 1-4, an ink formulation determines strength and a modulus of the hydrogel material of a prosthesis matrix; whereas a final “erection” deformation of the prosthesis is controlled by the designed bionic white membrane structure, and the size change is limited by the surface fibers, so that the matrix material does not affect the size. A fracture strain of the matrix material needs to be larger than an expansion strain of the designed prosthesis. On this basis, a final deformation size of the prosthesis maintains stable, and does not change with a change on a material type.

After the corpus cavernosum prosthesis structure prepared in embodiment 5 expands with water injection, the diameter can reach 1.4 times the original diameter, and the length becomes 1.6 times the original length. It shows that the deformation size of the prosthesis can be controlled by designing the fiber trajectory of the bionic white membrane.

After water is injected into the prosthesis structure prepared in comparative example 1, due to a lack of the fiber bionic white membrane structure, the structural deformation is uneven; a middle portion greatly deforms; and the prosthesis structure cannot achieve required hardness. Continued water injection will lead to an excessive deformation in a middle region of the prosthesis, to achieve a fracture strain point of the material, which results in a prosthesis damage. It shows necessity of designing a bionic white membrane.

After the water is injected into the prosthesis structure prepared in comparative example 2 for a period of time, the internal structure undergoes an uncontrollable deformation due to partial swelling, which results in a failure of the prosthesis structure. This is due to a lack of a secondary crosslinking step; and limited photocrosslinking sites are insufficient for formation of a more stable hydrogel structure in a water environment. This shows importance of secondary crosslinking of the prosthesis structure.

It can be seen from the above embodiments that the disclosure further provides a penile corpus cavernosum prosthesis. For the penile corpus cavernosum prosthesis provided by the disclosure, with biocompatible hydrogel as a matrix material, the mechanical properties are compatible with tissues, and the safety and the stability are good. An anticoagulant layer obtained by coating can effectively reduce the risk of thrombosis. The penile corpus cavernosum prosthesis of the disclosure has bionic vascular sinus cavity and bionic white membrane structures. Expansion with water injection corresponds to an erection state, which can make a penis reach a hardness and a size required for sexual life; and recovery with pressure relief corresponds to a flaccid state with reduced size and modulus, so that the penis maintains a more normal appearance. An initial size of the penile corpus cavernosum prosthesis of the disclosure can be customized by modeling, and a size of the erection and expansion state can be regulated by the bionic white membrane structure as needed. The prepared prosthesis can meet the requirements of personalized customization of a patient. 3D printing has a high precision structure, and is fast in formation and low in cost.

The descriptions above are just preferred implementations of the disclosure, it should be noted that several improvements and embellishments may also be made without departing from the scope and spirit of the disclosure to those ordinary skilled in the art, and these improvements and embellishments should also be considered to fall within the scope of the disclosure.

What is claimed is:

1. A bio-ink, prepared from the following raw materials in percentage by mass:
5-60% of a polymer-based material, 0.01-1% of an initiator, 0.05-10% of a crosslinking agent, 0.01-1% of a light absorber, and the balance of water.

2. The bio-ink according to claim 1, wherein the polymer-based material is one or more of gelatin, chitosan, sodium alginate, agarose, hyaluronic acid, silk fibroin, dextran, polyvinyl alcohol, polyethylene glycol, acrylamide, acrylic acid, N-isopropylacrylamide, methacrylic acid, benzyl methacrylate, polyvinyl alcohol methacryloyl, 2-hydroxyethyl methacrylate, and N-vinylpyrrolidone;
the initiator is one or more of 2-hydroxy-4-(2-hydroxyethoxy)-2-methylpropiophenone, lithium phenyl-2,4,6-trimethylbenzoylphosphinate, benzophenone, and 2,4-dimethylthioxanthone.

3. The bio-ink according to claim 1, wherein the crosslinking agent is one or more of N, N-methylenebisacrylamide, poly (ethylene glycol) diacrylate, and 4-arm poly (ethylene glycol) diacrylate;
the light absorber is one or more of hydroquinone, 1-(4-sulfophenyl)-4-(4-sulfophenylazo)-5-pyrazolone-3-carboxylic acid trisodium salt, 2,2′-dihydroxy-4, 4′-dimethoxybenzophenone-5, and 5′-sodium disulfonate.

4. An application of the bio-ink according to claim 1 in 3D printing.

5. A penile corpus cavernosum prosthesis, comprising a main body hydrogel structure, an internal bionic vascular sinus cavity structure, and an external bionic white membrane structure;
wherein the main body hydrogel structure is obtained by 3D printing with the bio-ink according to claim 1.

6. The penile corpus cavernosum prosthesis according to claim 5, wherein the internal bionic vascular sinus cavity comprises a cylindrical cavity structure and a spherical cavity structure;
a channel of the cylindrical cavity structure has a length of 1-100 mm, and a diameter of 0.1-10 mm;
the spherical cavity structure has a diameter of 0.5-5 mm.

7. The penile corpus cavernosum prosthesis according to claim 5, wherein a fiber material of the external bionic white membrane structure is high density polyethylene fibers;
each fiber of the external bionic white membrane structure has a diameter of 0.05-1 mm, and there are 10-40 fibers.

8. A preparation method for the penile corpus cavernosum prosthesis according to claim 5, comprising the following steps:
(1) performing 3D printing and curing on the bio-ink according to any one of claims 1-3 to obtain a main body model structure;
(2) embedding fibers into an outer surface of the main body model structure, and then performing light curing to obtain a main body model with a bionic white membrane structure; and
(3) performing crosslinking and coating on the main body model with the bionic white membrane structure to obtain the penile corpus cavernosum prosthesis.

9. The preparation method according to claim 8, wherein in step (1), for 3D printing, a printing resolution is 10-50 μm, a printing speed is 1-60 s/layer, a printing power is 600-1000 w, and a height of each layer is 40-60 μm;
in step (1), for curing, a wavelength of ultraviolet light is 280-450 nm, and a time lasts for 0.5-2 h;
in step (1), a trench of the main body model structure has a width of 0.1-2 mm, and a depth of 0.2-4 mm;
in step (2), for light curing, a wavelength of ultraviolet light is 280-450 nm, and a time lasts for 5-10 min.

10. The preparation method according to claim 8, wherein in step (3), a crosslinking solution for crosslinking is one or more of a glutaraldehyde solution, a 1-ethyl-(3-dimethylaminopropyl) carbodiimide/N-hydroxysuccinimide solution, a genipin solution, a calcium chloride solution, and a sodium hydroxide solution;
a mass percentage of the crosslinking solution is 0.1-20%;
in step (3), a crosslinking time lasts for 5-30 h;
in step (3), coating is to load poly-L-lysine and heparin sodium in sequence;
a coating thickness is 1-10 μm.
